(19) United States
(12) Patent Application Publication
(10) Pub. No.: US 2025/0099780 A1
(43) Pub. Date: Mar. 27, 2025

(54) DEVICE AND METHOD FOR TREATING A TISSUE

(71) Applicant: ALVIV LASER SOLUTIONS LTD., Tel Aviv-Yafo (IL)

(72) Inventors: Yair LEOPOLD, Raanana (IL); Shira DORON, Tel Aviv-Yafo (IL)

(73) Assignee: ALVIV LASER SOLUTIONS LTD., Tel Aviv-Yafo (IL)

(21) Appl. No.: 18/969,527

(22) Filed: Dec. 5, 2024

Related U.S. Application Data
(63) Continuation-in-part of application No. PCT/IL2023/050445, filed on May 1, 2023.
(60) Provisional application No. 63/349,594, filed on Jun. 7, 2022.
(60) Provisional application No. 63/606,620, filed on Dec. 6, 2023.

(51) Int. Cl. A61N 5/067; A61N 5/06

(52) U.S. Cl. CPC A61N 5/067; A61N 5/0603; A61N 2005/0608; A61N 2005/0626; A61N 2005/063

(57) ABSTRACT

A treatment device is disclosed. The treatment device comprising: a cover dimensioned for insertion into the anal sphincter area, connected to a handpiece; at least one optical fiber partially, configured to emit a laser beam, wherein the laser beam is directed perpendicularly to axis of the at least one optical fiber's; a laser source coupled to the at least one optical fiber; a thermometer connected to the cover; and a controller configured to control the laser source based on temperature measurements received from the thermometer.

### CROSS-REFERENCE TO RELATED APPLICATION

This application is a continuation in part of PCT Patent Application No. PCT/IL2023/050445 having International filing date of May 1, 2023, which claims the benefit of priority of U.S. Provisional Patent Application No. 63/349,594 filed on Jun. 7, 2022.

This application also claims the benefit of priority of U.S. Provisional Patent Application No. 63/606,620 filed on Dec. 6, 2023.

The contents of the above applications are incorporated herein by reference in their entirety.

### FIELD OF THE INVENTION

The present invention relates generally to laser-based medical treatment devices. More specifically, the present invention relates to laser-based medical treatment devices for treating a tissue.

### BACKGROUND OF THE INVENTION

The gastrointestinal (GI) tract, also called the alimentary canal, is a muscular hollow continuous tubular organ that starts at the mouth and terminates at the anus and is responsible for the digestion and absorption of ingested food and liquids. Circular rings of smooth muscles, called sphincters, surround portions of the alimentary canal. There are two circular muscles located in the anal canal, called the internal anal sphincter (IAS) and external anal sphincter (EAS) muscles. The IAS and EAS control the outflow of gas and fecal material from the anus. The IAS is a thin white muscle wrapped around the anal canal. The internal sphincter has a resting tightness which keeps small amounts of liquid and gas from escaping unexpectedly during rest and sleep. Wrapped around the IAS is the EAS; a large thick red voluntary muscle. The EAS can be used to voluntarily delay defecation. The EAS muscle is a voluntary muscle, and the IAS muscle is an involuntary muscle.

The internal anal sphincter (IAS) is an abridgment of the circular smooth muscular layer of the anal canal, typically about 5 mm thick and about 35 mm in length. It is responsible for approximately 65% of involuntary resting tone. The external anal sphincter (EAS) thickness is typically between 4 and 30 mm and is responsible for about 25% of the resting tone. Normally these muscles work in tandem to control the outflow of gas and fecal material from the anal canal through the anus. Anal sphincter muscles may become weak after having babies or with increasing age.

Weakened or damaged IAS or EAS muscles in the anal canal can cause sphincter dysfunction or a decrease in anal sphincter tone, such that they may leak gas, liquid feces, or solid feces, which can cause extreme embarrassment. This results in fecal incontinence also known as bowel incontinence; fecal material can descend through the anal canal without warning and stimulate the sudden urge to defecate. The resulting inability to control bowel movements, causes stool (feces) to leak unexpectedly from the anal canal. Fecal incontinence ranges from an occasional leakage of stool while passing gas to a complete loss of bowel control. The loss of dignity associated with losing control over one's bodily functions can lead to embarrassment, shame, frustration and depression. It's common for people with fecal incontinence to try to hide the problem or to avoid social engagements.

In certain surgical systems, radiofrequency energy is applied to tissue at different tissue levels to create multiple tissue lesions however patients treated for fecal incontinence with radiofrequency have reported high levels of pain due to electrode puncturing the rectal tissue with negligible long-term benefits. Furthermore, radiofrequency treatments are applied over small areas and require a manual repetitive handpiece movement over the anus and rectal anatomy, as well as inside the anal canal. Furthermore, this type of movement can be embarrassing for patients in a clinical setting, due to the rotation and in-and-out motion. Typical treatment temperatures are between 85 and 95 degrees Celsius while the typical adult rectal temperature can vary between 36 to 38 degrees Celsius. It may be advantageous to apply preheating to the anal canal and control the temperature of the tissue surrounding the anal canal to be closer to the treatment temperature in order to eliminate the need for electrodes to puncture the tissue. With preheating noninvasive radiofrequency treatment depths could be increased without the need for electrodes puncturing the tissue.

In other surgical systems laser energy is applied directly to tissue to create lesions. Laser treatments have been used for decades to rejuvenate the vaginal canal. So-called vaginal treatment can increase vaginal wall thickness and restore moisture to the tissues. It can reduce vaginal dryness, vaginal atrophy, and mild involuntary bladder leakage. Laser treatments can be painless however the laser's energy application mostly targets and heats the surface of the tissue, which is enough for treating the thin IAS but not enough to treat the thick EAS which wraps around the IAS. Due to the limited penetration of laser energy; lasers are not well suited to treat fecal incontinence. During a laser energy treatment much of the laser energy is dissipated into the surrounding tissue which is much cooler than the tissue being treated. This loss of energy occurs in an uncontrolled manner and limits the depth of treatment of the laser. By preheating the rectal tissue to just below treatment temperature, treatment depth of the laser can be increased with minimal laser energy in a more controlled fashion. Therefore, it would be advantageous to provide a system of controlling the rectal tissue temperature before applying laser energy. Temperature probes located on a handheld device could provide feedback to a temperature controller which could apply heating and cooling to control the temperature of the anal canal to the desired preheat temperature for example to 45° C. Furthermore, laser energy can be applied radially or semi-radially reducing the number of energy treatments and shortening the overall treatment time.

Furthermore, laser treatments have a more stationary handpiece, with a radial laser, to cover all areas of the rectal wall as it is withdrawn from the anal canal shortening the treatment time and eliminating the need for rotation or in-and-out motion. Application of laser energy is aided by adhering to a treatment protocol to ensure that the tissue is not heated beyond the treatment range and damaging or burning of tissue occurs. Application of laser energy can be further aided by monitoring of certain tissue and/or device parameters to ensure that the tissue is not heated beyond the treatment range and damaging or burning of tissue occurs.

Ultrasound therapy is a treatment long used by physical therapists or occupational therapists to relieve pain and to promote tissue healing. Ultrasound therapy can penetrate to the deeper tissue, However, there are many contraindications for ultrasound such as pregnancy, treatment near the spine, infection, ischemia and others. Focused ultrasound beams of ultrasonic energy can be applied precisely and accurately on deep tissues such as the EAS without damaging surrounding bone or tissue. Focused ultrasound energy can also provide beneficial nerve stimulation in and near the anal canal. By preheating the tissue in the anal canal and controlling the temperature to be closer to treatment temperature focused ultrasound can be applied even more precisely and accurately as the ultrasound energy will dissipate more slowly into surrounding tissues.

Preheating tissue prior to treatment has many benefits. Preheating can increase depth of treatment temperatures, it can provide a controlled temperature prior to treatment which increases precision and accuracy of treatment, and it can reduce the treatment energy dissipation into surrounding tissue. However, there are limitations such as thermal injury in the application of thermal energy. While a temperature of 45 degrees Celsius can be sustained for three hours before tissue damage occurs, 50 degrees Celsius can cause tissue injury after four minutes and 55 degrees Celsius can cause tissue injury after only 30 seconds, and 60 degrees Celsius can cause tissue injury after five seconds. Therefore, it would be advantageous to provide preheating for a controlled and limited amount of time just prior to treatment and then followed by cooling in order to protect the tissue from injury. A temperature control system which provides both heating and cooling is therefore ideal. Furthermore, a temperature control system which can both apply heating energy and absorb heat energy quickly is also advantageous. For example, a system which can increase the rectal temperature from 37 degrees Celsius to 50 degrees Celsius for approximately 2 minutes before laser energy is applied following which the system can cool the rectal temperature back to 45 degrees Celsius or even to 37 degrees Celsius. There are a number of methods to apply such heating and cooling. Preheating could be achieved through applying laser energy which converts to heat energy. Preheating could be achieved through the use of ultrasound energy which converts to heat energy. Preheating could be achieved through the use of radiofrequency energy which converts to heat energy. A resistive heating device, an inductive heating device, or an infrared heating device could preheat tissue. Heated air, water, or other fluids could be channeled through the anal canal in order to preheat the tissue. Similarly, air, water, or other fluids could be used to cool the anal canal. There are many ways to heat and cool tissue that are well known, the previous examples of heating and cooling methods are not exhaustive.

However, none of the known methods provides precise control of the treated tissue temperature, prior, during and/or after the laser treatment.

### SUMMARY OF THE INVENTION

Some aspects of the invention may be directed to a treatment device, comprising: a cover dimensioned for insertion into the anal sphincter area, connected to a handpiece; at least one optical fiber partially, configured to emit a laser beam, wherein the laser beam is directed perpendicularly to axis of the at least one optical fiber's; a laser source coupled to the at least one optical fiber; at least one holder for holding the at least one optical fiber at a fixed distance, of between 2 to 15 mm, from inner walls of the cover; at least one thermometer connected to the cover; and a controller configured to control the laser source based on temperature measurements received from the thermometer.

In some embodiments, the at least one optical fiber is a radial emission fiber. In some embodiments, the at least one optical fiber is a side-fire fiber and the device further comprises an actuator configured to rotate the side-fire fiber around the fiber's axis. In some embodiments, the device further comprises one or more optical elements configured to direct the laser beam to be perpendicular to the optical fiber's axis. In some embodiments, the one or more optical elements are selected from, lenses, mirrors, prisms, and splitters.

In some embodiments, the device further comprises a sleeve inserted into the cover, at least partially encompassing the optical fiber, wherein the sleeve comprises an optically transparent portion configured to allow the delivery of the laser beam to the anal sphincter area during treatment. In some embodiments, the optically transparent portion comprises at least one of: an optically transparent material and one or more windows allowing the delivery of the laser beam.

In some embodiments, the cover comprises an optically transparent portion configured to allow the delivery of the laser beam to the anal sphincter area during treatment. In some embodiments, the optically transparent portion comprises at least one of: an optically transparent material and one or more windows allowing the delivery of the laser beam.

In some embodiments, wherein the thermometer is located at a distal end of the cover away from the handpiece. In some embodiments, the device further comprises an axial movement element for axially sliding the sleeve inside the cover.

In some embodiments, the device further comprises a heating unit configured to provide heat to the anal sphincter area during treatment. In some embodiments, the controller is further configured to control the heating unit based on the temperature measurements received from the thermometer. In some embodiments, the controller is configured to: receive the temperature measurements; control the heating unit to heat to the anal sphincter area until the anal sphincter area reaches a desired temperature; and control the laser source to generate the laser beam only after the target temperature was reached. In some embodiments, the controller is further configured to: continuously receive temperature measurements; terminate the laser generation if the temperature exceeds a predefined threshold temperature. In some embodiments, the heating unit is selected from, radiofrequency (RF) electrodes attached to the cover, an ultrasound prob located in the cover, and a resistance wire located in the cover.

In some embodiments, the device further comprises at least one pressure sensor located on the external surface of the sleeve.

Some additional aspects of the invention may be directed to a method for treating a sphincter tissue of a patient comprising: inserting a treatment device into the patient's anal sphincter area; measuring a temperature of a sphincter tissue at at least one location; and radiating the patient's anal sphincter area with a laser beam based on the temperature measurements. In some embodiments, the treatment device comprises: a cover dimensioned for insertion into the anal sphincter area, connected to a handpiece; at least one optical fiber partially, configured to emit the laser beam, wherein the laser beam is directed perpendicularly to the optical axis of the at least one optical fiber; a laser source coupled to the at least one optical fiber; a thermometer connected to the cover; and a controller configured to control the laser source based on temperature measurements received from the thermometer.

In some embodiments, the method further comprises: heating the sphincter tissue to a desired temperature; and wherein radiating comprises radiating the patient's anal sphincter area only when the temperature of the sphincter tissue reached the target temperature.

In some embodiments, the method further comprises: monitoring the temperature of the sphincter tissue at the at least one location; and terminating the laser radiation if the temperature exceeds a predefined threshold temperature.

In some embodiments, the method further comprises: receiving pressure measurements from the anal sphincter area; and determining a state of fecal incontinence of the patient based on the received pressure measurements. In some embodiments, the pressure measurements are received from one or more pressure sensors included in the treatment device.

### BRIEF DESCRIPTION OF THE DRAWINGS

The subject matter regarded as the invention is particularly pointed out and distinctly claimed in the concluding portion of the specification. The invention, however, both as to organization and method of treatment, together with objects, features, and advantages thereof, may best be understood by reference to the following detailed description when read with the accompanying drawings in which:

FIG. 1 is an anatomic view of the rectum and anal canal;

FIGS. 2A and 2B are illustrations of a treatment device according to some embodiments of the invention;

FIG. 3A is an illustration of another treatment device according to some embodiments of the invention;

FIGS. 3B and 3C are illustrations of a perspective and exploded views of another treatment device according to some embodiments of the invention;

FIGS. 3D and 3E are illustrations of a perspective and exploded views of a cover connected to a handpiece according to some embodiments of the invention;

FIGS. 3F and 3G are illustrations of detachably connectable thermometers according to some embodiments of the invention;

FIGS. 4A, 4B, and 4C are illustrations of laser optical fiber tips according to some embodiments of the invention; and

FIG. 5 is a flowchart of a method of treating a tissue according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

Some aspects of the invention are directed to various laser-based systems and methods for treating the dysfunction of sphincters and adjoining tissue regions in the body. For example, such systems and methods may be particularly well suited for treating these dysfunctions in the lower gastrointestinal tract, e.g., in the intestines, rectum and anal canal. It should be appreciated that the disclosed systems and methods are applicable for use in treating other dysfunctions elsewhere in the body, e.g., for restoring compliance to or otherwise tightening interior tissue or muscle regions. The systems and methods that embody features of the invention are also adaptable for use with systems and Surgical techniques that are not necessarily laser-based.

### Anatomy of the Rectum and Anal Canal

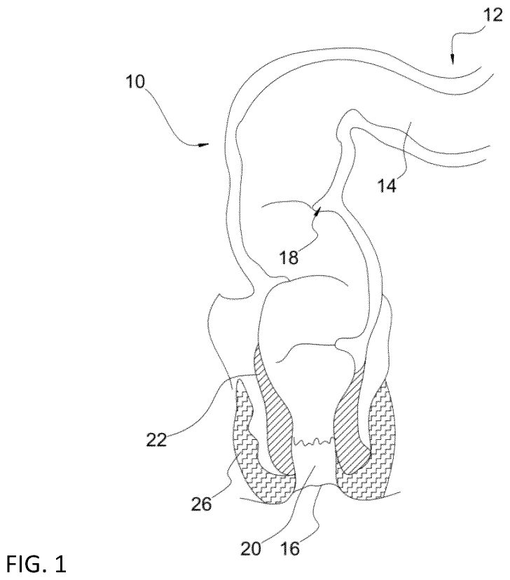

Reference is now made to FIG. 1 which shows the anatomy of the rectum and anal canal. The rectum is the terminal part of the large intestine 12. The rectum 10 extends from the sigmoid flexure 14 (which is the narrowest part of the colon) to the anal orifice 16. The rectum 10 is about 15 to 17 cm in overall length. The upper or superior portion of the rectum 10 extends downward from the sigmoid flexure 14. This portion of the rectum 10 is almost completely surrounded by the peritoneum. A mucous membrane lines this portion of the rectum 10. The mucous membrane is thicker, of a darker color, and more vascular than elsewhere in the colon. The superior portion of the rectum 10 contains a number of permanent folds of a semilunar shape, which are called the Houston valves 18.

As FIG. 1 shows, there are usually three Houston valves 18. Sometimes a fourth is present, and occasionally only two are found. When rectum 10 is empty, the Houston valves 18 overlap each other. The valves 18 support the weight of fecal matter, to slow its descent toward the anal orifice 16. When the inferior or lower part of the rectum 10 is contracted to expel fecal matter, a number of additional folds develop in the mucous membrane of the Superior portion of rectum 10, to urge fecal matter downward. The middle portion of rectum 10 is covered anteriorly and laterally by the peritoneum as it extends from the Superior portion. However, as rectum 10 extends further downward, the lateral peritoneum gradually recedes. The lower or inferior portion of the rectum 10 is called the anal canal 20. It typically extends about 4 to 5 cm above the anal orifice 16. The anal canal 20 is invested by the internal sphincter muscle 22, and surrounded at its termination by the external sphincter muscle 26.

### Treatment Device

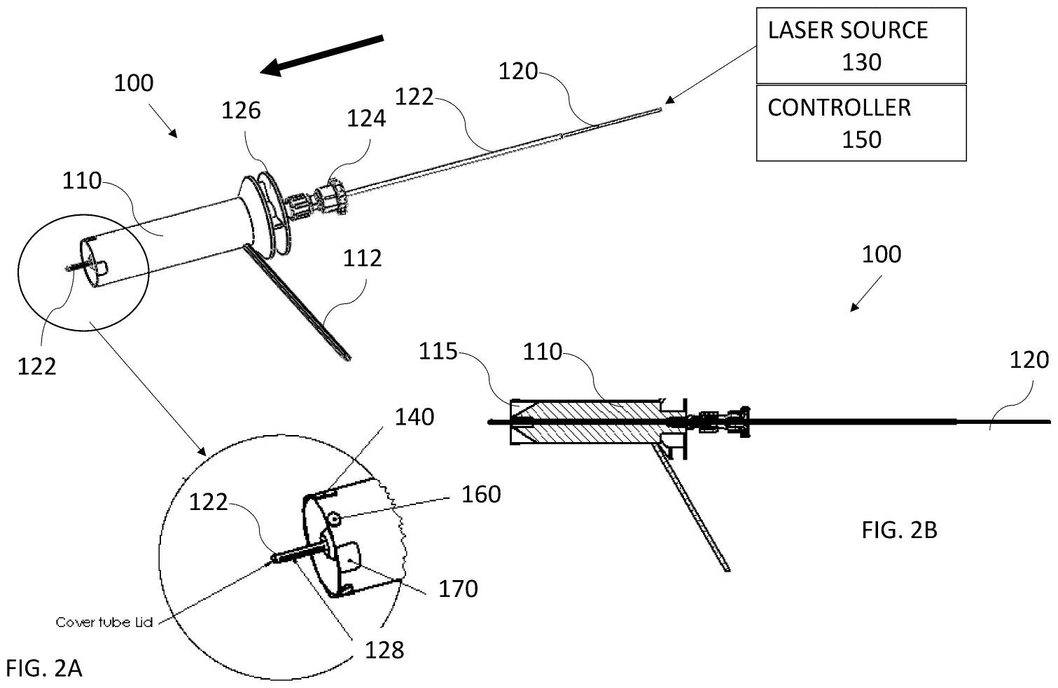

Reference is now made to FIGS. 2A and 2B which are illustrations of a perspective and cross-section views of a treatment device according to some embodiments of the invention. A treatment device 100 may be configured to provide a controlled laser treatment to tissues such as, for example, the sphincter muscle. Device 100 may include a cover 110 dimensioned for insertion into a body cavity, such as, the rectum 10 through the anal orifice 16 and the sphincter muscles 22 and 26 areas. Cover 110 may be connected to a handpiece 112. Cover 110 may be made or may include any medical grad polymer, metal or alloy. In some embodiments, cover 110 may comprise an optically transparent portion configured to allow the delivery of the laser beam to the anal sphincter area during treatment. In some embodiments, the optically transparent portion comprises at least one of: an optically transparent material and one or more windows allowing the delivery of the laser beam.

Handpiece 112 may be configured to be held by a user (e.g., a physician) and may be configured to allow manipulation of device 100 during use thereof.

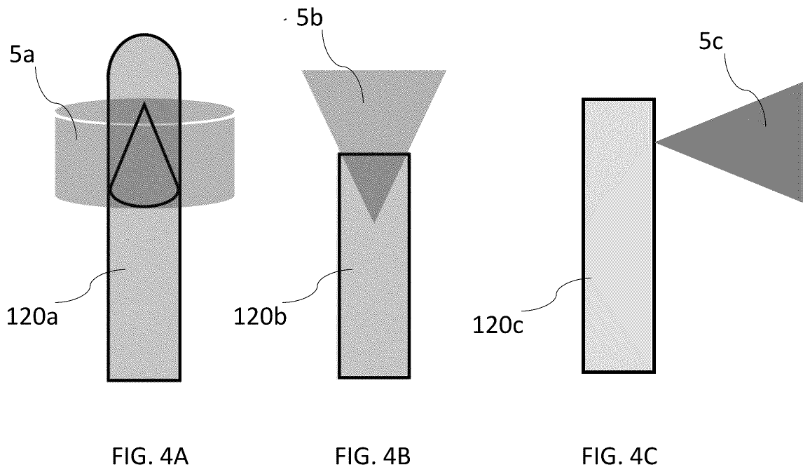

In some embodiments, device 100 may further include at least one optical fiber 120, inserted into cover 110, for example, slidably inserted into cover 110. In some embodiments, optical fiber 120 may be coated/covered by a sleeve or may be a bear fiber. Optical fiber 120 may be configured to emit a laser beam. In some embodiments, the laser beam may be directed perpendicularly to the optical axis of the at least one optical fiber's. Some nonlimiting examples of such optical fibers are given and discussed with respect to FIGS. 4A, 4B and 4C. Sleeve 122 may at least partially encompass and protect fiber 120. Without limitation, the laser energy can be transmitted in a fiber optics with a diameter of 300-1000 μm. Optical fibers 120 may be, without limitation: a side-firing fiber which can emit the energy at angles such as 70-90° from the optical axis; a radial fiber, which emits light at 360° from the optical axis; or a diffused end portion fiber, in which light is scattered around the distal end, so that the light-emitting pattern of the diffusing fiber exhibits a uniform distribution along the distal end (e.g., the diffused distal part may be 0.5-4 cm in length and emits the light at 360° along the diffused length).

In some embodiments, device 100 may further include a sleeve 122 slidably inserted into cover 110 and at least one optical fiber 120 at least partially inserted into sleeve 122. Sleeve 122 may be made or may include any medical grad polymer, metal or alloy. In some embodiments, sleeve 122 may include an optically transparent portion 128 configured to allow the delivery of the laser beam to the anal sphincter area during treatment. In some embodiments, optically transparent portion 118 comprises an optically transparent material and one or more windows or openings allowing the delivery of a laser beam therethrough. For example, sleeve 122 and/or portion 128 may be made from a transparent polymer. In yet another example, sleeve 122 may be made from an opaque material and may include one or more windows or openings allowing the delivery of a laser beam therethrough.

In some embodiments, device 100 may further include one or more holders 115 for holding optical fiber 120 and/or sleeve 122 (encompassing optical fiber 120) in a fixed distance from the walls of cover 110, for example, between 2 to 15 mm, between 5 to 7, between 4 to 10 mm, between 6 to 11 mm, between 9 to 13 mm, between 10 to 15 mm or any value or range in-between. Therefore, holders 115 may ensure keeping a safe distance between optical fiber 120 and the tissue of for example, an anal canal.

Device 100 may further include a laser source 130 coupled to at least one optical fiber 120. Laser source 130 may include any source configured to generate a medical laser. The laser source may include a pumping laser diode, a laser cavity, one or more mirrors, and any additional required optical element. The laser source may generate a continuous laser or a pulsating laser. For example, laser source 130 may generate a pulsating laser at a wavelength of between 700 to 2200 nm. The wavelengths can be individual wavelengths or a combination of wavelengths, such as but not limited to: 980 nm (medium depth of penetration), 1064 nm (greater depth of penetration) or 1470 nm (more superficial depth of penetration). All of these wavelengths can be achieved with a diode laser with a power of 5-60 watts (continuous or pulsed).

Device 100 may further include a thermometer 140 connected to cover 110. In the nonlimiting example illustrated in the enlarged section of FIG. 2A, thermometer 140 is located at a distal end of cover 110 away from handpiece 112. Thermometer 140 may be configured to measure the temperature of the tissue, for example, the anal sphincter tissue. In some embodiments, two or more thermometers 140 may be attached to different locations along cover 110. In some embodiments, one or more thermometers 140 may be configured to measure the temperature of the anal sphincter tissue prior to the application of the laser treatment, during the application of the laser treatment and/or after application of the laser treatment, for example, periodically as defined (e.g., every 30 sec) or continuously in real-time mode. Thermometer 140 may include any suitable temperature sensors, such as, a thermocouple, an infrared (IR) based sensor, and the like.

Device 100 may further include a controller 150 configured to control laser source 130 based on temperature measurements received from thermometer 140. Controller 150 may include a processing unit, and a memory for storing instructions to be executed by the processing unit, for example, instructions to control laser source 130 based on temperature measurements received from thermometer 140. Controller 150 may further include a communication unit for communicating with laser source 130 and thermometer 140.

In some embodiments, device 100 may further include an axial movement element 124 for axially sliding sleeve 122 inside cover 110. For example, the user may manipulate sleeve 122 back and forth inside cover 110, thereby changing the location of fiber 120 tip. In some embodiments, device 100 may further include a dilator 126.

In some embodiments, device 100 may further include a heating unit 160 configured to provide heat to the anal sphincter area during treatment. In a nonlimiting example, heating unit 160 may include one or more radio-frequency (RF) electrodes attached to cover 110 (as illustrated), for example, at the distal end of cover 110. Some additional nonlimiting examples may include an ultrasound prob located in cover 110, a resistance wire located in cover 110, and the like.

In some embodiments, controller 150 may be configured to control heating unit 160 based on the temperature measurements received from thermometer 140.

For example, controller 150 may be configured to: receive the temperature measurements; control heating unit 160 to heat the anal sphincter area until the anal sphincter area reaches a desired temperature between 38 to 45° C. (e.g., 42° C.); and control laser source 130 to generate the laser beam only after the target temperature was reached. In some embodiments, controller 150 may further be configured to continuously receive temperature measurements from thermometer 140 and terminate the laser generation if the temperature exceeds a predefined threshold temperature between 42 to 46° C. (e.g., 44° C.).

In some embodiments, device 100 may further include at least one pressure sensor 170 located on the external surface of cover 110, for measuring the pressure applied by external sphincter muscle 26. For example, the pressure may be measured prior to the application of the laser treatment and after the termination of laser treatment in order to estimate the ongoing effectiveness of the treatment. In some embodiments, parameters of the laser generation may be determined based on the pressure measurements. In some embodiments, the pressure measurements may help in the decision of whether another laser treatment cycle is required and/or regarding the duration of the laser application.

In some embodiments, cover 110 may hold one or more communication and/or power supply cables for providing electrical power to at least one of, thermometer 140, heating unit 160 and pressure sensor 170 and to send/receive communication signals from and to at least one of, thermometer 140, heating unit 160 and pressure sensor 170.

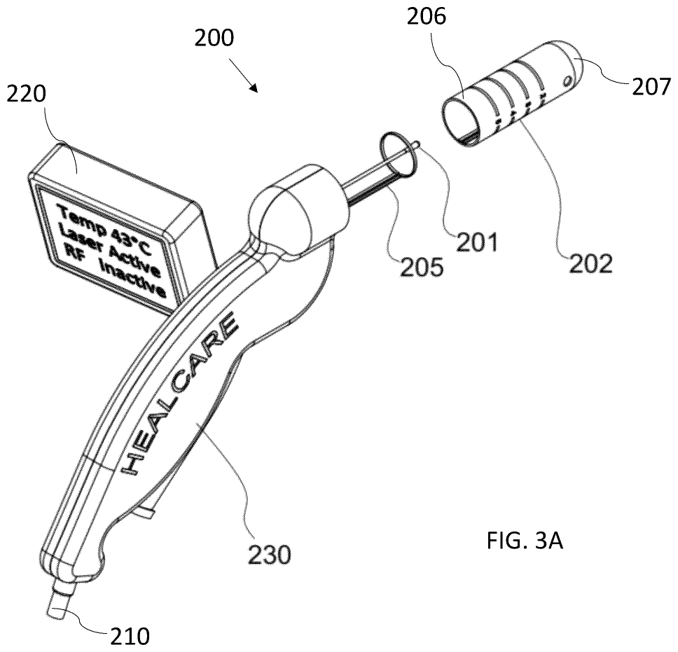

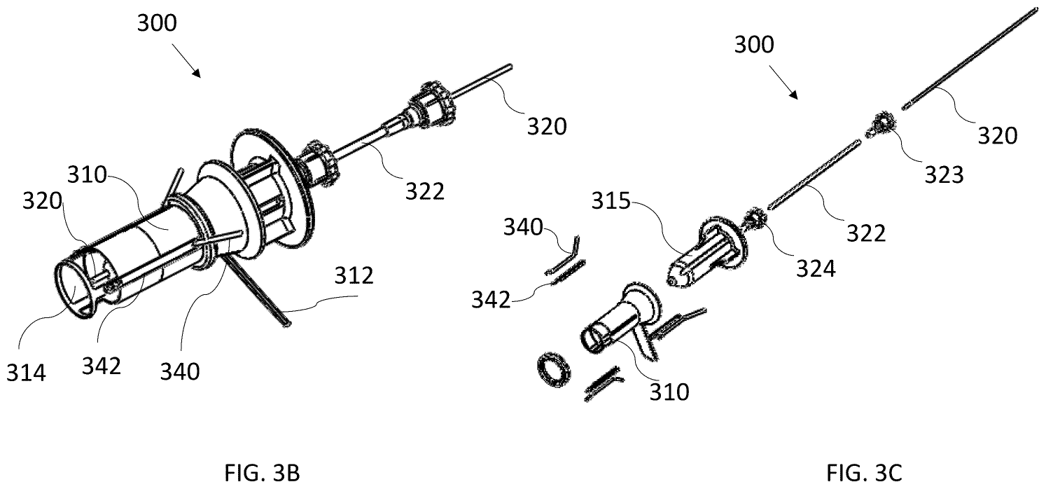

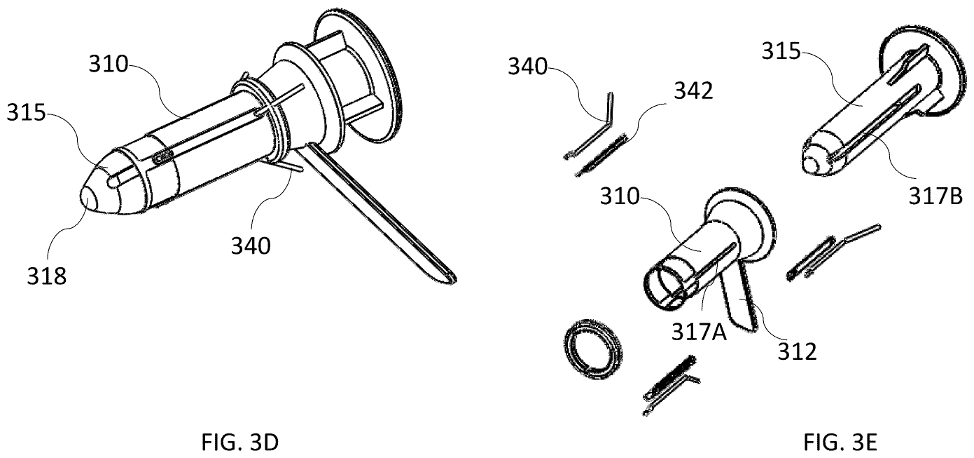

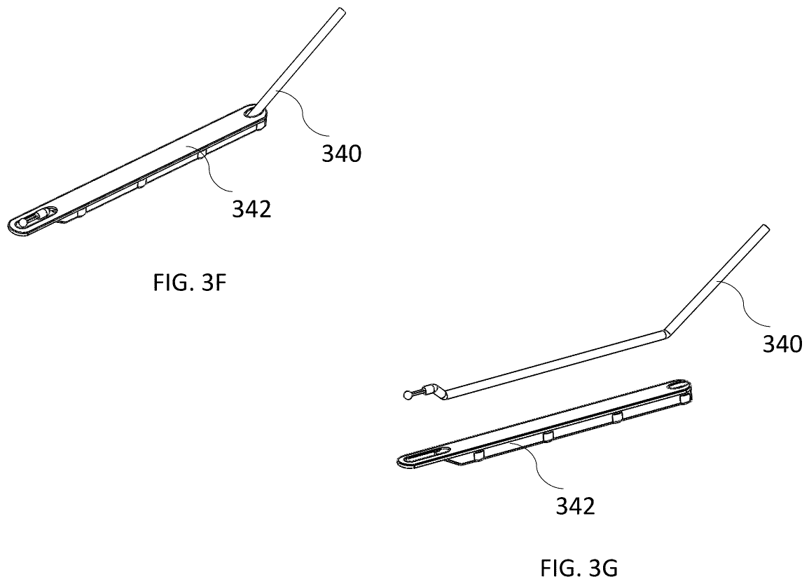

Reference is now made to FIG. 3A which is an illustration of another treatment device according to some embodiments of the invention. FIG. 3A shows a handheld portion of a device for treating dysfunction of the external sphincter muscle 26, or internal sphincter muscle 22, or both. In FIG. 3A, device 200 may include a handpiece made, e.g., from molded plastic. The handpiece 230 is sized to be conveniently held by a physician, in the same fashion as an anoscope. A monitoring screen 220 may be attached/available nearby so that the user can monitor temperatures during the procedure. It should be appreciated that monitor screen 220 may be located on handpiece 230 as illustrated but may also be located in any other place visible to the physician during treatment. In some embodiments, monitoring screen 220 may communicate or may be included in a controller, such as controller 150.

Device 200 may also include a cover 202 (e.g., a transparent barrel) having a distal end 206. A thermometer (e.g., a temperature probe) 203 may be located near introducer 207 (e.g., at least partially transparent introducer having a bullet-shape) of cover 202. In the nonlimiting example, illustrated in FIG. 3 bullet-shaped introducer 207 extends a distance beyond the distal end 206. Cover 202 and introducer 207 are sized (e.g., by having a maximum outside diameter of about 30 mm to 33 mm) for insertion into the rectum 10 through the anal orifice 16. The introducer 207 aids passage through the anal canal 20.

Device 200 may further include an optical fiber 201 (e.g., a radial laser fiber) and a resistive heating element 205. The device 200 is connected to auxiliary components through channel 210. Auxiliary components may provide energy, heating, cooling, control signals and feedback.

Reference is now made to FIGS. 3B and 3C which are illustrations of a perspective and exploded views of another treatment device according to some embodiments of the invention. A device 300 may include a cover 310 dimensioned for insertion into a body cavity, such as, the rectum through the anal orifice and the sphincter muscles areas. Cover 310 may be connected to a handpiece 312. Cover 310 may be made or may include any medical grad polymer, metal or alloy. In some embodiments, cover 310 and handpiece 312 may be fabricated as a single piece. In some embodiments, cover 310 may comprise one or more windows 314 allowing the delivery of a laser beam.

Device 300 may further include at least one optical fiber 320, inserted into cover 310, for example, slidably inserted into cover 310. In some embodiments, optical fiber 320 may be coated/covered by a sleeve 322 or may be a bear fiber. Optical fiber 320 may be configured to emit a laser beam. In some embodiments, the laser beam may be directed perpendicularly to the optical axis of the at least one optical fiber 320. Some nonlimiting examples of such optical fibers are given and discussed with respect to FIGS. 4A, 4B and 4C. Sleeve 322 may at least partially encompass and protect fiber 320. In some embodiments fiber 320 may be inserted into sleeve 322 via two luer-locks 323 and 324.

In some embodiments, device 300 may include a holder 315, insertable into cover 310, as detailed in FIGS. 3C and 3D. Holder 315 may be configured to hold at least one optical fiber 320 (or sleeve 322 encompassing fiber 320) at a fixed distance of between 2 to 15 mm (and any value in between) from inner walls of cover 310. For example, holder 315 may include a centered outlet 318 (shown in FIG. 3D) from which a distal end of fiber 320 exits, as shown in FIG. 3B. In some embodiments, holder 315 may hold the distal end of fiber 320 at a fixed distance of between 2 to 15 mm (and any value in between) from windows 314. Therefore, holders 315 may ensure keeping a safe distance between the distal end optical fiber 320 and the tissue of for example, an anal canal.

In some embodiments, device 300 may further include a detachably connectable thermometer 340 attached to cover 310. In some embodiments, detachably connectable thermometer 340 comprising detachably connected thermometer housing 342, to be detachably connected from cover 310, and optionally also sleeve 315. Therefore, cover 310, and optionally also sleeve 315 may include corresponding slits 317A and 317B. Slits 317A and 317B are configured to receive and secure thermometer housing 342, and are better illustrated in FIGS. 3F and 3G.

In some embodiments, cover 310 and/or holder 315 may be for a single use, and may be replaced prior to every new treatment. Therefore, detachably connected thermometer 340 may be detached from cover 310 and/or holder 315 after a first used and reattached to a new cover 310 and/or holder 315 for another use.

In some embodiments, device 300 may include any one of the additional elements included in device 100 and/or 200, such as, but not limited to heating unit 160 and controller 150.

Reference is now made to FIGS. 4A, 4B, and 4C which are illustrations of various optical fiber tips and the shape of the laser beam emitted from each tip. FIG. 4A shows radial emission fiber 120a that is configured to emit a radial laser beam 5a perpendicular to fiber 120a optical axis. FIG. 4B is an illustration of a bare fiber configured to emit laser beam 5b in the direction of fiber 120b optical axis. In order to direct laser beam 5b in a direction perpendicular to fiber 120b optical axis, one or more optical elements may be assembled in device 100. For example, the one or more optical elements may be selected from, lenses, mirrors, prisms, splitters and the like.

FIG. 4C is an illustration of a side-fire fiber 120c configured to fire laser beam 5c perpendicularly to fiber 120a optical axis. In such an arrangement, device 100 may further include an actuator (not illustrated) configured to rotate the side-fire fiber around the fiber's axis, thereby providing a circular treatment of the anal sphincter area.

### Method of Treating a Sphincter Tissue

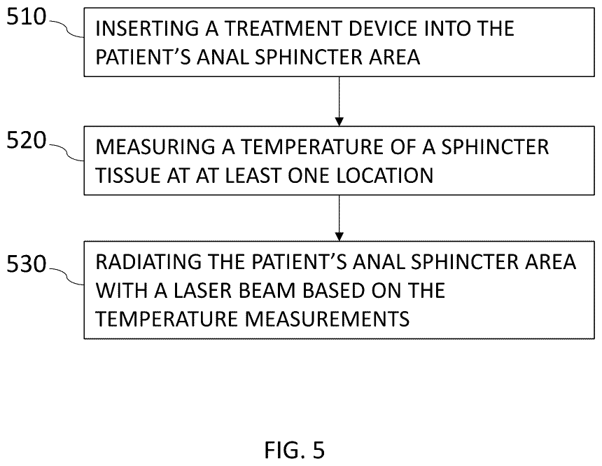

Reference is now made to FIG. 5 which is a flowchart of a method of treating a sphincter tissue of a patient according to some embodiments of the invention. The method of FIG. 5 may be performed by a user (e.g., a physician, the patient) using any one of devices 100 or 200.

In step 510, a treatment device may be inserted into the patient's body cavity, such as the anal sphincter area. For example, a physician may insert treatment device 100 or 200 into the anal sphincter area of a patient, e.g., suffering from fecal incontinence.

In step 520, a temperature of a sphincter tissue at least one location, is measured, for example, using thermometer 140.

In step 530, the patient's anal sphincter area may be radiated with a laser beam based on the temperature measurements. For example, a laser beam may be generated by laser source 130 and emitted from the tip of optical fiber 120 at a direction perpendicular to optical fiber 120 axis, as to radiate the anal sphincter area. In some embodiments, the laser source is moved based on marks throughout the sphincter length under temperature control.

In some embodiments, prior to radiating the tissue with the laser beam, the sphincter tissue may be heated to desired temperature between 38 to 45° C. (e.g., 42° C.). In some embodiments, the desired temperature may be determined based on data related to the patient, such as, age, gender, the severity of the medical condition (e.g., fecal incontinence), and the like. In some embodiments, radiating the patient's anal sphincter area is initiated only when the temperature of the sphincter tissue reaches the target temperature.

In some embodiments, the method may further include monitoring the temperature of the tissue at the at least one location; and terminating the laser radiation if the temperature exceeds predefined threshold temperature between 42 to 46° C. (e.g., 44° C.).

In some embodiments, the method may further include receiving pressure measurements from the anal sphincter area and determining a state of fecal incontinence of the patient based on the received pressure measurements. For example, pressure applied by the sphincter muscle (e.g., internal sphincter muscle 22) may be measured using pressure sensor 170. In some embodiments, the state of fecal incontinence may be used for determining the treatment parameter (e.g., energy provision, duration, etc.) of the laser beam.

In some embodiments, various treatment devices disclosed in this specification can be supplied to a physician as part of a sterile kit. The kit packages the particular treatment device as a single use item in a sterile fashion within peripherally sealed sheets of plastic film material that are torn or peeled away at the instance of use. The kit can include, together with the particular treatment device or separately supplied, instructions for using the device according to one or more of the methodologies disclosed herein.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A treatment device, comprising:
a cover dimensioned for insertion into the anal sphincter area, connected to a handpiece;
at least one optical fiber partially, configured to emit a laser beam, wherein the laser beam is directed perpendicularly to axis of the at least one optical fiber's;
a laser source coupled to the at least one optical fiber;
at least one holder for holding the at least one optical fiber at a fixed distance, of between 2 to 15 mm, from inner walls of the cover;
at least thermometer connected to the cover; and
a controller configured to control the laser source based on temperature measurements received from the thermometer.

2. The treatment device of claim 1, wherein the at least one optical fiber is a radial emission fiber.

3. The treatment device of claim 1, wherein the at least one optical fiber is a side-fire fiber and the device further comprises an actuator configured to rotate the side-fire fiber around the fiber's axis.

4. The treatment device of claim 1, further comprising one or more optical elements configured to direct the laser beam to be perpendicular to the optical fiber's axis.

5. The treatment device of claim 4, wherein the one or more optical elements are selected from, lenses, mirrors, prisms, and splitters.

6. The treatment device of claim 1, further comprising a sleeve inserted into the cover, at least partially encompassing the optical fiber, and wherein the holder is configured to hold the sleeve at the fixed distance of between 2 to 15 mm, from the inner walls of the cover.

7. The treatment device of claim 6, wherein the sleeve comprises an optically transparent portion configured to allow the delivery of the laser beam to the anal sphincter area during treatment.

8. The treatment device of claim 6, wherein the optically transparent portion comprises at least one of: an optically transparent material and one or more windows allowing the delivery of the laser beam.

9. The treatment device of claim 1, wherein the cover comprises an optically transparent portion configured to allow the delivery of the laser beam to the anal sphincter area during treatment.

10. The treatment device of claim 9, wherein the optically transparent portion comprises at least one of: an optically transparent material and one or more windows allowing the delivery of the laser beam.

11. The treatment device of claim 1, wherein the thermometer is located at a distal end of the cover, away from the handpiece.

12. The treatment device of claim 1, wherein the thermometer is detachably connected to the cover.

13. The treatment device of claim 1, further comprising an axial movement element for axially sliding the sleeve inside the cover.

14. The treatment device of claim 1, further comprising a heating unit configured to provide heat to the anal sphincter area during treatment.

15. The treatment device of claim 14, wherein the controller is further configured to control the heating unit based on the temperature measurements received from the thermometer.

16. The treatment device of claim 14, wherein the controller is configured to:
a. receive the temperature measurements;
b. control the heating unit to heat to the anal sphincter area until the anal sphincter area reaches a desired temperature; and
c. control the laser source to generate the laser beam only after the target temperature was reached.

17. The treatment device of claim 16, wherein the controller is further configured to:
d. continuously receive temperature measurements;
e. terminate the laser generation if the temperature exceeds a predefined threshold temperature.

18. The treatment device of claim 14, wherein the heating unit is selected from, radiofrequency (RF) electrodes attached to the cover, an ultrasound prob located in the cover, and a resistance wire located in the cover.

19. The treatment device of claim 1, further comprising at least one pressure sensor located on the external surface of the sleeve.

20. A method for treating a sphincter tissue of a patient comprising:
inserting a treatment device into the patient's anal sphincter area;
measuring a temperature of a sphincter tissue at at least one location; and
radiating the patient's anal sphincter area with a laser beam based on the temperature measurements,
wherein the treatment device comprises:
a cover dimensioned for insertion into the anal sphincter area, connected to a handpiece;
at least one optical fiber partially, configured to emit the laser beam, wherein the laser beam is directed perpendicularly to the optical axis of the at least one optical fiber;
a laser source coupled to the at least one optical fiber;
a thermometer connected to the cover; and
a controller configured to control the laser source based on temperature measurements received from the thermometer.
